(19) United States
(12) Patent Application Publication
(10) Pub. No.: US 2009/0197973 A1
(43) Pub. Date: Aug. 6, 2009

(54) Method for preparing emulsion or dispersion, and foodstuff, skin externals and medicaments containing emulsion or dispersion obtained by the method

(75) Inventors: Jun Arakawa, Kanagawa (JP); Hisahiro Mori, Kanagawa (JP); Tomohide Ueyama, Kanagawa (JP)

Correspondence Address: Moss & Burke, PLLC, 401 Holland Lane, Suite 407, Alexandria, VA, 22314, US

(73) Assignee: FUJIFILM CORPORATION, Minato-ku (JP)

(21) Appl. No.: 12/320,557

(22) Filed: Jan. 29, 2009

(30) Foreign Application Priority Data
Feb. 1, 2008 (JP) 2008-023278

(51) Int. Cl. A61K 47/06; A61P 17/00; A61P 43/00

(52) U.S. Cl. 514/772; 426/602

(57) ABSTRACT

The present invention provides that a method for preparing an emulsion or a dispersion composed of an oily phase and an aqueous phase by contacting the oily phase with the aqueous phase, the method comprising: injecting the oily phase, which contains a water-soluble organic solvent and at least one hydrophobic functional ingredient and in which the content of a surfactant is approximately 0.1% by mass or less based on the total mass of the oily phase, into the aqueous phase such that the Reynolds number of the oily phase immediately before contact with the aqueous phase is approximately 1,000 or more, and that foodstuffs, a skin external and a medicament which contain an emulsion or a dispersion prepared by the method described above.

### CROSS REFERENCE TO RELATED APPLICATION

This application claims priority under 35 USC 119 from Japanese Patent Applications No. 2008-023278, the disclosure of which is incorporated by reference herein.

### BACKGROUND OF THE INVENTION

1. Field of the Invention

The present invention relates to a method for preparing an emulsion or a dispersion, and foodstuff, a skin external and a medicament containing the emulsion or the dispersion obtained by this preparation method.

2. Description of the Related Art

It has been a common practice to extract hydrophobic functional ingredients from natural animals and plants or cell wall of yeasts having been proliferated by the fermentation method with the use of water-soluble organic solvents such as alcohols of the like and then add the same to foods and drinks as food additives or to cosmetics as functional cosmetic ingredients. Owing to the recent rising concern about health, attempts have been vigorously made to extract ingredients from various natural materials. Active ingredients are extracted with alcohols from materials that have been discarded as wastes, for example, banana leaves, citrus residues and outer onion skin.

In general, these hydrophobic functional ingredients extracted with water-soluble organic solvents such as alcohols are obtained as powders, oils or waxes of the hydrophobic functional ingredients from a concentration/drying step of removing the organic solvent from the extract, then used as starting materials for preparing foodstuff or drinks. To use these hydrophobic functional ingredients, most of which are hardly soluble in water, in water-based compositions such as water-based foods, water-based drinks or water-based cosmetics having a favorable texture, it is required solubilize the hydrophobic functional ingredients by using other solvents or oil or fats or melt the hydrophobic functional ingredients per se at a high temperature followed by an emulsifying or dispersing step before adding to the water-based compositions. Thus, an increase in the production cost is unavoidable. To effectively absorb a hydrophobic functional ingredient, in particular, a size of hydrophobic functional ingredient oil droplets is required to decrease under 1 μm or less and, therefore, much energy is required for the emulsification. In addition, there sometimes arises a problem that, in the course of once drying a hydrophobic functional ingredient and then dissolving or melting the same, the hydrophobic functional ingredient is denatured and causes a decrease in the ingredient content or the occurrence of some side effects due to the denatured ingredient.

In the case of emulsifying or dispersing by a method which comprises adding emulsifier to an aqueous organic solvent solution containing a hydrophobic functional ingredient and dividing oil droplets into smaller droplets in the aqueous phase by a commonly employed emulsification method of applying a strong shear force from outside, it is known that a system containing a large amount of a water-soluble organic solvent can be hardly emulsified. In this case, furthermore, the particle diameter distribution becomes broad and large particles are separated within a short time. As a result, foodstuff or cosmetics containing such an emulsion or a dispersion has insufficient keeping qualities.

EP-A No. 1180062 discloses a method for continuously producing micro or nanoparticles of a natural material using a micromixer. In this method, particle-forming phase containing a natural active ingredient and an aqueous phase containing water as the main component are respectively fed into microchannels and periodically mixed together each in the form of a layered liquid, thereby particulating the natural active ingredient. In this method, however, the average particle diameter of the particles thus formed exceeds 1000 nm and, therefore, clearly far from the target level of 200 nm.

JP-A No. 2001-340738 discloses a method of dispersing glycosylceramide with an ultra-high pressure emulsifying machine using a jet stream. However, the particle side of the particles dispersed in the dispersion obtained by this method still exceeds 200 nm.

JP-A No. 2005-103421 discloses a method wherein a solution prepared by dissolving silicone oil and a surfactant in a water-soluble solvent is directly injected into an aqueous phase at a speed of 300 m/min or above without coming into contact with the outside environment. However, this method is effective exclusively for silicone oil in practice. Namely, it is unsuitable for hydrophobic materials extracted from animals or plants. In addition, a large amount of a surfactant is employed in this method, which is unfavorable from the viewpoint of the side effects of the surfactant.

With respect to the preparation of organic nanoparticles, on the other hand, studies have been made on the gas phase method (a method comprising sublimating a sample in an inert gas atmosphere and then collecting particles on a substrate), the liquid phase method (for example, a reprecipitation method comprising injecting a sample dissolved in a good solvent into a poor solvent under stirring or temperature-controlling to give microparticles), the laser ablation method (a method comprising laser irradiating a sample dispersed in a solution and miniaturizing the particles by ablation) and so on. Preparation examples attempting to monodisperse particles of a desired size by these methods have been reported.

Among all, the reprecipitation method disclosed in JP-A Nos. 6-79168 and 2006-341242 or the like is noteworthy as a method for producing organic nanoparticles that is excellent in convenience and productivity. In this method, a solution of an organic material dissolved in a good solvent is injected into a stirring tank filled with a poor solvent through a liquid supply port such as a nozzle or a tube. The contents of the stirring tank are stirred with a stirring means such as a magnetic stirrer, a three-one motor, a disperser or a lamond stirrer. The organic material is exposed to the poor solvent immediately after the injection into the stirring tank and, therefore, crystal nuclei are formed and grow to nanocrystals within a short time. As a typical example thereof, an organic nanoparticle dispersion having nanocrystals dispersed in an aqueous solvent can be cited. This method is disclosed as a method for preparing an inkjet pigment dispersion or a color filter pigment dispersion in the patent documents as cited above and so on.

Different from synthetic materials such as pigments, many hydrophobic functional ingredients extracted from animals or plants are in the form of oils or waxy fats which are little crystallizable. Even in the case of a crystallizable hydrophobic functional ingredient, crystallization scarcely arises immediately after transferring the molten matter into the poor solvent. Thus, favorable nanoparticles of hydrophobic functional ingredients extracted from animals or plants can be hardly prepared merely by employing the reprecipitation method disclosed in JP-A Nos. 6-79168 and 2006-341242.

As discussed above, it has been urgently required to develop a method for preparing an emulsion or a dispersion which contains microparticles emulsified or dispersed therein and suffers from little deterioration with the passage of time during preservation and an emulsion or a dispersion obtained thereby.

### SUMMARY OF THE INVENTION

The present invention has been made in view of the above circumstances and provides a method for preparing an emulsion or a dispersion, and a foodstuff, a skin external and a medicament containing the same.

A first aspect of the invention provides a method for preparing an emulsion or a dispersion composed of an oily phase and an aqueous phase by contacting the oily phase with the aqueous phase, the method comprising: injecting the oily phase, which contains a water-soluble organic solvent and at least one hydrophobic functional ingredient and in which the content of a surfactant is 0.1% by mass or less based on the total mass of the oily phase, into the aqueous phase such that the Reynolds number of the oily phase immediately before contact with the aqueous phase is 1,000 or more.

A second aspect of the invention provides an emulsion or a dispersion prepared by the preparation method described above.

A third aspect of the invention provides the method for preparing an emulsion or a dispersion as described above, in which the hydrophobic functional ingredient is a functional food ingredient.

A fourth aspect of the invention provides a foodstuff which contains an emulsion or a dispersion prepared by the preparation method as described above.

A fifth aspect of the invention provides the method for preparing an emulsion or a dispersion as described above, in which the hydrophobic functional ingredient is a skin external ingredient.

A sixth aspect of the invention provides a skin external which contains an emulsion or a dispersion prepared by the preparation method as described above.

A seventh aspect of the invention provides the method for preparing an emulsion or a dispersion as described above, wherein the hydrophobic functional ingredient is a medicinal ingredient.

An eighth aspect of the invention provides a medicament which contains an emulsion or a dispersion prepared by the preparation method as described above.

### DETAILED DESCRIPTION OF THE INVENTION

The preparation method according to the invention is a method for preparing an emulsion or a dispersion composed of an oily phase and an aqueous phase by contacting the oily phase with the aqueous phase, the method comprising: injecting the oily phase, which contains a water-soluble organic solvent and at least one hydrophobic functional ingredient and in which the content of a surfactant is 0.1% by mass or less based on the total mass of the oily phase, into the aqueous phase such that the Reynolds number of the oily phase immediately before contact with the aqueous phase is 1,000 or more.

According to this preparation method, an emulsion or a dispersion containing a hydrophobic functional ingredient and fine particles dispersed therein and having a high preservation stability, can be obtained by regulating the surfactant content of the oily phase to 0.1% by mass or less based on the total mass of the oily phase and controlling the Reynolds number when injecting the oily phase into the aqueous phase (immediately before contact with the aqueous phase) so as to be 1,000 or more.

The “dispersion” as used herein means “a solid dispersion” or “a dispersion of solid matters” comprising solid particles dispersed in a dispersion medium, which solid is obtained mainly by emulsification, dispersion or a post-treatment.

The terms “hydrophobic functional ingredient” or “functional foodstuff ingredient” as used herein mean a hydrophobic ingredient or a foodstuff ingredient as a result of which a certain physiological effect may be expected to be induced in a living body when the ingredient is applied to or introduced into the living body.

Additionally, in the invention, the term “step” indicates not only an independent step but may also indicate a step which cannot be discriminated clearly from other steps, as long as the intended effects of the step may be obtained.

Further, any notation for expressing numerical ranges in the invention indicates a range defined by the minimum and maximum values and includes the minimum and maximum values.

The invention will be described below.

The hydrophobic functional ingredient constituting the emulsified particles or dispersed particles in the emulsion or the dispersion according to the invention means an ingredient having 1% by mass or less, preferably 0.5% by mass or less and still preferably 0.1% by mass or less, solubility in water at 25° C.

In the invention, the hydrophobic functional ingredient may be one originating in an animal or a plant, one originating in a fossil fuel such as petroleum, coal or natural gas, or a synthetic or semi-synthetic product obtained from such a material, without specific restriction. It is particularly preferable to use a hydrophobic functional ingredient extracted from an animal or a plant using an organic solvent.

Extraction means one of chemical separation procedures whereby a liquid or solid material is brought into contact with a solvent and an ingredient contained in the material that is soluble in the solvent is selectively separated from ingredients that are insoluble or hardly soluble in the solvent. The extraction procedure to be performed in the invention falls within the category of the general extraction procedure as described above, though the material used therein is an animal- or plant-origin solid and a water-soluble solvent is employed as the extraction solvent.

The animal or plant materials usable in the invention are not particularly restricted. Supposing that the emulsion or dispersion of the invention is used mainly in health foods and health drinks as the final products, plant materials are preferred to animal materials from the viewpoints of odor, taste and healthy image. With respect to the parts of plants, any parts including leaf, stem, peel and fruit are usable as the material. Furthermore, algae, hyphae, yeasts and fungi are also preferable.

Examples of land plants usable herein include Angelica keiskei, Phaseolus angularis, Gynostemma pentaphyllum, alfalfa, aloe, ginkgo, Urtica thunbergiana, Azadirachta indica, Indian gooseberry, Foeniculum vulgare (fennel), Curcuma longa (turmeric), Prunus mume, Citrus unshu, Echinacea purpurea, Acanthopanax senticosus, Sambucus nigra (elder), Astragalus membranaceus, Plantago asiatica, Onopordum, Hordeum vulgare (barley), green barley leaf, Abelmoschu esclentus, Panax ginseng, oat wheat, Olea europaea (olive), Diospyros kaki, Curcuma zedoaria (zedoary), Valeriana fauriei, Chamomilla recutita, Paullinia cupana, Glycyrrhiza glabra (licorice), Garcinia cambogia, Aloe arborescens, Gymnema sylvestre, Uncaria tomentosa (cat's claw), Allium victorialis, Psidium guajava, Lycium chinense, Pueraria lobata, Sasa veitchii, Orthosiphon aristata (Curnisctin), Vaccinium macrocarpon (cranberry), Citrus grandis (grapefruit), Morus alba (mulberry), Cinnamomum cassi, Laurus nobilis, Brassica oleracea var acephala (kale), Gentiana lutea, Kothala himbutu (Salacia reticulata), Coffea arabica (coffee), Sesamum indicum (sesame), Oryza sativa (rice), Triticum vulgare (wheat), Amorphophallus konjac (elephant foot), Punica granatum (pomegranate), Crocus sativus (saffron crocus), Crataegus cuneata, Panax notoginseng, Perilla ocymoides [Perilla frutenscens acuta], Pterocarpus santalinus (Dalbergia cochinchinensis), Jasminum officinale (jasmine), Betula platyphylla, Zingiber officinale (ginger), Equisetum arvense (field horsetail), Stevia rebaudiana, Hypericum perforatum (St. John's wort), Crataegus oxyacantha, Prunus domestica, Taraxacum officinale, Aesculus hippocastanum (marronier), Polygonum fagopyrum [Fagopyrum esculentum], Glycine soja (soybean), Citrus aurantium (better orange), Thymus vulgaris (thyme), Tamarindus indica, Allium cepa (onion), Aralia elata Seemann (Japanese angelica tree), chaste tree, Thea sinensis (tea), Cynara scolymus (artichoke), camellia, Centella asiatica, Rubus suavissimus, Capsicum annuum, Houttuynia cordata, Eucommia ulmoides, Solanum lycopersicum (tomato), Daucus carota sativa (carrot), Phoenix dactylifera (date), Allium sativum (garlic), Serenoa repens (Saw palmetto), Tabebuia impetiginosa (Pau d'arco), Nelumbo nucifera, Carum petroselinum (parsley), Coix lacryma-jobi ma-yuen (Job's tears), Capsicum annuum cv (paprika), rose, Vaccinium myrtitllus, Eriobotrya japonica (loquat), Tussilago farfara (coltsfoot), Vitis binefera (grape), black cohosh, blueberry, propolis, Carthamus tinctorius (safflower), Spinacia oleracea (spinach), Peumus boldu, Lepidium meyenii (maca), macadamia nuts, Manchurian wild rice, pine, Ilex paraguariensis (mate), Tagetes spp. (marigold), mandarin orange, Acer nikoense Maximowicz (Nikko Maple), Oenothera biennis (evening primrose), Melissa officinalis (melissa), Melilotus officinalis Lam, Corchorus olitorius L. (Mulukhiyya), yucca, Artemisia princeps (mugwort), Apocynum venetum L. (Luobuma), Lavandula angustifolia (lavender), Pyrus malus (apple), Litchi chinensis (lychee), Citrus medica lemonum (lemon), Rosmarinus offisinalis (rosemary), Walteria indica, moss plants, fern plants and the like, though the invention is not restricted thereto.

With respect to algae, all photosynthetic algae generating oxygen are usable herein. Examples of the algae include cyanobacteria, Prochlorophyta, Glaucophyceae, Rhodophyceae, Prasinophyceae, Ulvophyceae, Chlorophyceae, trebouxiophyceae, Charophyceae, Cryptophyceae, Chlorarachniophyceae, Euglenophyceae, Dinophyceae, Chrysophyceae, Raphidophyceae, Eustigmatophyceae, Xanthophyceae, Phaeophyceae, Cacillariophyceae, Dictyochophyceae, Pelagophyceae, Haptophyceae and the like. Among these algae, spirulina belonging to Cyanophyceae; Haematococcus belonging to Chlorophyceae; and Nemacystus, Laminaria, Undaria (seaweed) and Fucus (Kelp) belonging to Phyaeophyceae are particularly important materials.

There are various kinds of hyphae, yeasts and fungi. Examples thereof include Agaricus, yeasts, Lentinus edodes (Berk.) Sing (shiitake), Agaricus bisporus (champignon), Cordyceps sinensis (Cordyceps), Bacillus subtilis (Hay bacillus), Bifidobacteria, Monascus purpureus (Red yeast rice), Tremella fuciformis, Grifola frondosa (Gray-maitake), bisporus (common mushroom), Phellinus linteus, Hericium erinaceus, Ganoderma lucidum. Karst (Reishi) and the like, though the invention is not restricted thereto.

Various ingredients over a broad range can be extracted from these natural materials. As typical examples thereof, lipid ingredients contained in animals and plants are cited. As the lipid ingredients, there can be enumerated fatty acids, glycerides, complex lipids, terpenoids, steroids, prostaglandins and the like.

Among these lipid ingredients, examples of the active ingredient include linoleic acid, γ-linolenic acid, arachidonic acid, α-linolenic acid, eicosapentaenoic acid, docosahexaenoic acid, γ-aminobutyric acid, thioctic acid and the like. Examples of the glycerides include monoacylglycerol, diacylglycerol, triacylglycerol and the like. Examples of the complex lipids include phospholipids such as phosphatidic acid, phosphatidylcholine, phosphatidylethanolamine, phosphatidylserine and phosphatidylinositol; sphingo lipids such as sphingosine and sphingomyeline; and glycolipids such as monogalactosyl glyceride and glucosyl ceramide.

Examples of the active ingredients of terpenoids include monoterpenes such as linalool, citronellal, myrcene, limonene, pinene, menthol, cineol, camphor, longifolene, cedrol and caryophyllene; diterpenoids such as phytol, abietic acid, kaurene and gibberellin; triterpenoids such as squalene, dammarenediol and ursolic acid; tetraterpenoids typified by carotenoid pigments; and polyterpenoids such as gutta-percha, vitamin K2, ubiquinone and vitamin K1. Among these substances, carotenoid pigments may attract particular attention owing to the antioxidative activity thereof. Typical examples of the carotenoid pigments include astaxanthin, lycopene, zeaxanthin, lutein, capsanthin, fucoxyanthin, α-carotene and β-carotene.

Examples of the active ingredients of the steroids include sterols such as squalene, cholesterol, ergosterol, stigmasterol, dehydrocholesterol, cholecalciferol (vitamin D3) and 25-hydroxy vitamin D3; sex hormones such as testosteron, androsteron, progesterone, estrogen and estradiol; adrenal cortex hormones such as cortisone and dexamethasone; cardiac glycosides such as digitoxigenin, digoxigenin and gitoxigenin; steroid sapogenins such as diosgenin and cortisone; and bile acids such as cholic acid and deoxycholic acid.

As one of important active ingredients other than lipid ingredients, polyphenol can be cited. Polyphenol, which is a generic name for plant ingredients having two or more phenolic hydroxyl groups per molecule, constitutes plant pigments and bitter ingredients formed by photosynthesis, and has particularly excellent antioxidation activity. Examples of Polyphenols include flavonoids, chlorogenic acids, gallic acids, ellagic acids, lignans, curcumins and coumarins. Examples of flavonoids include isoflavones such as genistein and daizen; flavonols such as quercetin, kaempferol, myricetin and lutin; flavanons such as hesperidin and naringenin; anthocyanins such as cyanidin and delfinidin; flavanols such as epicatechin, epigallocatechin, epicatechin gallate and theaflavin; and flavones such as chrysin, apigenin and luteolin. Many of polyphenols have attracted public attention as ingredients of health foods and cosmetics and examples of such polyphenols may include galangin, fisein, chalcon, puerarin and resveratrol.

Other active ingredients may include alkaloids that are particularly useful in the medicinal field. Examples of the alkaloids include aconitine, atropine, ephedrine, caffeine, capsaicin, quinine, curare, cocaine, colchicine, scopolamine, strychnine, solanine, taxine, theophylline, dopamine, nicotine, vinca, berberine, morphine, lycorine and the like.

Further other active ingredients may include shgaol and gengerol extracted from ginger, allyl isothiocyanate that is contained in horseradish or mustard, and allicin, allin and scordine contained in garlic, and the like.

The water-soluble organic solvent to be used in the invention means an organic solvent which has a solubility in water at 25° C. of 10% by mass or more. From the viewpoint of the stability of the obtained emulsion or dispersion, the solubility in water is preferably 30% by mass or more, still preferably 50% by mass.

Examples of the water-soluble organic solvent include methanol, ethanol, 1-propanol, 2-propanol, 2-butanol, acetone, tetrahydrofuran, acetonitrile, methyl ethyl ketone, dipropylene glycol monomethyl ether, methyl acetate, methyl acetoacetate, N-methylpyrrolidone, dimethyl sulfoxide, ethylene glycol, 1,3-butanediol, 1,4-butanediol, propylene glycol, diethylene glycol, triethylene glycol and the like, and mixtures thereof. Among them, ethanol, propylene glycol and acetone are preferred in the case of being limited to the application to foodstuffs. In the case being limited to the application of cosmetics, ethanol and 2-propanol are preferred. Ethanol is particularly preferred as the water-soluble organic solvent.

In the extraction, either a single water-soluble organic solvent or a mixture of two or more water-soluble organic solvents may be used. It may be also possible to use a mixture of a water-soluble organic solvent with water. In the case of using a mixture with water, it may be preferable that the mixture contains at least 50% by volume, more preferably 70% by volume or more, of the water-soluble organic solvent as described above.

The active ingredient can be extracted from an animal or plant material by using the water-soluble organic solvent in accordance with a method commonly employed in the art. In general, a finely ground solid material is used and extraction is conducted at a temperature of, for example, 0° C. to 300° C.

The water-soluble organic solvent solution (hereinafter, sometimes referred to merely “oily phase” or “water-soluble organic solvent phase”) is substantially free from a surfactant. That is to say, the content of a surfactant in the oily phase is approximately 0.1% by mass or less based on the total mass of the oily phase, more preferably approximately 0.01% by mass or less from the view point of the stability of the particles in the obtained emulsion or dispersion.

In emulsification methods of disrupting large oil droplets such as agitation emulsification or high-pressure emulsification, it is a common practice to add a surfactant to the oily phase. This is because the surface tension should be lowered and thus large droplets should be disrupted into small droplets by orienting the surfactant as quickly as possible on the interface formed by the large deformation of the droplets, and it is overwhelmingly advantageous to supply a surfactant from the oily phase so as to shorten the diffusion distance. To disrupt large droplets into small droplets, the surfactant should be dispersed at an extremely high speed and it is, therefore, necessary to provide a large surfactant concentration gradient. Thus, the surfactant is required in an amount exceeding the adsorption level to the droplets formed. This excessive surfactant decreases the stability after the emulsification due to the Ostwald growth or the like. When such an emulsion is used in foods or cosmetics, moreover, it may cause an unfavorable taste, skin irritation and the like.

In the invention, in contrast thereto, the oily phase ingredients are once completely dissolved in the water-soluble organic solvent such as ethanol and then brought into contact with the poor solvent under definite conditions to thereby condense out and form particles. Therefore, no surfactant is needed in forming the microparticles. In addition, in contrast, for example, to other hydrocarbons derived from petroleum or the like, the hydrophobic functional ingredient that has been extracted from an animal or a plant can itself be oriented in an aqueous phase to a certain degree so that the microparticles thus formed can be maintained in a stable state. As a result, it becomes substantially unnecessary to add a surfactant, which would be disadvantageous from the viewpoint of the stability of the particles after the emulsification and the like, to the oily phase. In the case where a surfactant is required to improve the stabilization of the emulsion or dispersion, it is enough to merely add the surfactant in such an amount as being necessary for the interfacial adsorption.

To prevent a structural change of a hydrophobic additive that is finely dispersed in the aqueous phase, the oily phase may contain at least one natural cooperative additive. Examples of the cooperative additive include lipids selected from the group consisting of sphingolipids, cholesterols and phospholipids.

Sphingolipids are complex lipids containing sphingoids as a long-chain base component. In addition to sphingolipids that are so-called ceramides and phytosphingolipids, examples of the sphingolipids include sphingophospholipids (for example, sphingomyelin, sphingoethanolamine, or the like), sphingo glycolipid (for example, cerebroside, ganglioside, or the like), and sphingosin and phytosphingosin that are ceramide precursors, and the like. Moreover, glycerolipids containing glycerol as a substitute for sphingoid also fall within this category.

Phospholipids include glycerophospholipids and lysophospholipids in which a fatty acid bonded to the 1-position and/or to the 2-position of glycerophospholipid is lost. There are various kinds of phospholipids depending on the type of the compound bonded to the phosphate moiety of the phospholipid and examples thereof include phosphatidylcholine, phosphatidylethanolamine, phosphatidylinositol, phosphatidylserine, phosphatidic acid, phosphatidylglycerol and the like. In general, these phospholipids are provided in the form of lecithin extracted from animals or plants. In addition to such highly pure lecithin, use can be also made of hydrogenated lecithin, enzymatically decomposed lecithin, enzymatically decomposed and hydrogenated lecithin, hydroxylecithin or the like.

Examples of the sterols include cholesterol, phytosterols (β-sitosterol, campesterol, stigmasterol, brassicasterol and the like), cholestenone, phytostenone, cholesterol esters, phytosterol esters, cholesterol glycosides phytosterol glycosides and the like.

Although any one of the above-described lipids may be used as the lipid to be added to the oily phase in the invention, sphingo lipids, sphingo glycolipids and phytosphingosine may be particularly preferred from the viewpoint of the stability of the emulsified/dispersed state. Either one of these lipids or a mixture of two or more kinds of the same may be used in the invention.

From the viewpoint of the stability of the emulsion or dispersion, the content of the lipid may range from approximately 0.1% by mass to approximately 30% by mass, more preferably from approximately 1% by mass to approximately 10% by mass.

The aqueous phase of the invention, i.e., the aqueous solution, is a solution comprising water as the main component.

The aqueous phase may contain a nonionic surfactant, an ionic surfactant, a water-soluble salt, a saccharide, a polysaccharide, a protein, a pH controlling agent, an antioxidant, a preservative, a colorant, a perfume or the like as will be described hereinafter.

Examples of the ionic surfactant include alkyl sulfonic acid salts, alkyl sulfuric acid salts, monoalkyl phosphonic acid salts, fatty acid salts, lecithin and the like. Examples of the salt include sodium chloride, sodium citrate, sodium ascorbate and the like. Examples of the saccharide include glucose, fructose, sucrose, arabinose, cellobiose, lactose, maltose, trehalose and the like. Examples of the polysaccharide include maltdextrin, oligosaccharides, inulin, acacia, chitosan and the like. Examples of the protein include various amino acids, oligopeptides, gelatin, water-soluble collagen, casein, cyclodextrin and the like.

As the pH controlling agent, use can be made of a base such as sodium hydroxide, an acid such as hydrochloric acid or a buffer solution such as a phosphate buffer solution or a citrate buffer solution. Examples of the antioxidant include ascorbic acid, ascorbic acid derivatives, citric acid monoglyceride and the like.

The amount of these additives can be controlled to 20% by mass or less, preferably 10% by mass or less, based on the total mass of the aqueous phase. If necessary, a small amount of a water-soluble organic solvent may be preliminarily added to the aqueous phase. In this case, the water-soluble organic solvent is added in an amount of 20% by mass or less, preferably 10% by mass or less, based on the total mass of the aqueous phase by taking the stability of the emulsion or dispersion into consideration.

The aqueous phase may contain a nonionic surfactant having an HLB of 10 or more but not more than 16 (hereinafter sometimes merely called “nonionic surfactant”) to improve the dispersibility.

From the viewpoint of stabilizing the dispersion, the nonionic surfactant usable in the invention is preferably a water-soluble nonionic surfactant. The water-soluble nonionic surfactant is not specifically restricted, so long as being a nonionic surfactant which is soluble in water.

As described above, it is preferable that this nonionic surfactant has an HLB of 10 or more but not more than 16, more preferably 12 or more but not more than 16 from the viewpoint of the stability of the emulsion or dispersion.

The HLB used herein is the balance of hydrophilicity-hydrophobicity generally used in the field of surfactants. It can be calculated by using a generally used calculation formula such as Kawakami's calculation formula.

In the invention, the following Kawakami's calculation formula is employed.

HLB=7+11.7 log(Mw/Mo)

In the above formula, Mw is the molecular weight of the hydrophilic group, and Mo is the molecular weight of the hydrophobic group.

Alternatively, use can be made of HLB values listed in, for example, manufacturers' catalogs. As is apparent from the above formula, a nonionic surfactant having an optional HLB value can be obtained by utilizing additive properties of HLB.

Examples of the nonionic surfactant suitably usable in the invention include (mono, di, tri) glycerol fatty acid esters, monoglycerol organic acid esters, polygrycelol fatty acid esters, propylene glycol fatty acid esters, polyglycerol condensed ricinoleic acid esters, sorbitan fatty acid esters, sucrose fatty acid esters and the like. Among these nonionic surfactants, polyglycerol fatty acid esters, sucrose fatty acid esters or combinations thereof may be more preferred from the viewpoint of improving the stability of the dispersion. Either one of these nonionic surfactants or a combination of two or more kinds thereof at an arbitrary ratio may be used. The nonionic surfactant is not always required to be a highly purified one by, for example, distillation or the like, and may be a reaction mixture.

Examples of the polyglycerol fatty acid ester usable in the invention include an ester of a polyglycerol having an average degree of polymerization of 4 or more, preferably from 6 to 10, and a fatty acid having from 8 to 18 carbon atoms such as caprylic acid, capric acid, lauric acid, myristic acid, palmitic acid, stearic acid, oleic acid or linoleic acid. Preferable examples of the polyglycerol fatty acid ester include hexaglycerol monopalmitate, hexaglycerol monomyristate, hexaglycerol monolaurate, decaglycerol monooleate, decaglycerol monostearate, decaglycerol monopalmitate, decaglycerol monomyristate and decaglycerol monolaurate.

Those polyglycerol fatty acid esters can be used alone or as mixtures thereof.

Examples of suitable commercially available products include NIKKOL Hexaglyn 1-L, NIKKOL Hexaglyn 1-M, NIKKOL Decaglyn 1-L, NIKKOL Decaglyn 1-M, NIKKOL Decaglyn 1-SV, NIKKOL Decaglyn 1-50SV, NIKKOL Decaglyn 1-ISV, NIKKOL Decaglyn 1-O, NIKKOL Decaglyn 1-OV and NIKKOL Decaglyn 1-LN that are products of Nikko Chemicals Co., Ltd.; RYOTO polyglyesters L-10D, L-7D, M-10D, M-7D, P-8D, S-28D, S-24D, SWA-20D, SWA-15D, SWA-10D and O-15D that are products of Mitsubishi-Kagaku Foods Corporation; and POEM J-0381V and POEM J-0021V that products of Riken Vitamin Co., Ltd.

The sucrose fatty acid ester to be used in the invention is preferably one having 12 or more, more preferably from 12 to 20, carbon atoms in the fatty acid moiety.

Preferable examples of the sucrose fatty acid ester include sucrose monooleate, sucrose monostearate, sucrose monopalmitate, sucrose monomyristate, sucrose monolaurate and the like.

In the invention, those sucrose fatty acid esters can be used alone or as mixtures thereof. Examples of commercially available product of the sucrose fatty acid ester include RYOTO sugar esters S-1170, S-1170S, S-1570, S-1670, P-1570, P-1670, M-1695, O-1570, OWA-1570, L-1695 and LWA-1570 that are products of Mitsubishi-Kagaku Foods Corporation; DK esters F140, F160 and SS that are products of Daiichi-Kogyo Seiyaku Co., Ltd; and the like, though the invention is not particularly restricted thereto.

The nonionic surfactant is added in an amount of preferably approximately 0.01 to approximately 10% by mass, more preferably approximately 0.05 to approximately 5% by mass and still more preferably approximately 0.1 to approximately 2% by mass based on the total mass of the emulsion or dispersion. Based on the hydrophobic functional ingredient, the amount of the nonionic surfactant is preferably from approximately 0.1 to approximately 100% by mass, more preferably approximately 1 to approximately 10% by mass.

From the standpoint of elevating the stability of the emulsion or dispersion, it may be preferable that the content of the nonionic surfactant in the aqueous phase is approximately 0.1% by mass or more. It may be also preferable to control the amount of the nonionic surfactant to approximately 2% by mass or less, since troubles such as vigorous foaming of the emulsion or dispersion scarcely arise in this case.

(Jet Injection Method)

The preparation method according to the invention comprises injecting the oily phase described above into the aqueous phase such that the Reynolds number thereof immediately before contact with the aqueous phase is approximately 1,000 or more.

When attempting to form nanoparticles of the hydrophobic functional ingredient using a different mechanism from those using pigments, dyes or the like, the inventors found that by jet-injecting the oily phase composed of the respective ingredients discussed above into the aqueous phase under conditions such that the Reynolds number is approximately 1,000 or more, nanoparticles of the hydrophobic functional ingredient can be formed and thus a fine emulsion or a fine dispersion can be obtained even in a case in which the hydrophobic functional ingredient does not crystallize.

When nanoparticles of a functional material are formed by a dispersion method commonly employed in foods, cosmetics or medicaments, a surfactant needs to be employed in a large amount. In contrast, according to the method of the invention wherein the oily phase is injected into the aqueous phase under the conditions defined above, it is possible to use none or a greatly reduced amount of the surfactant, which is very advantageous.

The injection method described above will be referred to as a “jet injection method” hereinafter.

The Reynolds number Re used herein is a non-dimensional number represented by the following formula:

Re=DUρ/μ

wherein Re represents the Reynolds number; D represents the inner diameter (m) of a tube or nozzle for injecting the oily phase; U represents the cross-sectional average velocity (m³/sec) of the oily phase; p represents the density (kg/m³) of the oily phase; and μ represents the viscosity (kg/m sec) of the oily phase.

When the oily phase is injected into the aqueous phase through a tube having a non-circular cross-sectional shape, the hydraulic-equivalent diameter Deq is employed as a substitute for D. The hydraulic-equivalent diameter Deq is provided in accordance with the following formula:

Deq=4A/L

wherein Deq represents the hydraulic-equivalent diameter; A represents the cross sectional area of the flow; and L represents the wet perimeter length.

In the invention, the Reynolds number of the oily phase at the moment of contact with the aqueous phase is approximately 1,000 or more. From the viewpoint of microparticulation, the Reynolds number is preferably approximately 1,500 or more. However, from the viewpoint of equipment costs, etc., it is preferably approximately 10,000 or less. Thus, it is particularly preferable that the Reynolds number ranges from approximately 2,000 to approximately 10,000. When the Reynolds number is approximately 1,000 or less, the emulsified or dispersed particles cannot be sufficiently microparticulated and thus the advantages of the invention cannot be achieved. When the Reynolds number exceeds approximately 10,000, on the other hand, it becomes necessary to use, for example, a high-pressure pump for feeding the oily phase, which is not practical from the standpoints of equipment costs and power consumption.

From the viewpoint of stability, the addition flow rate of the oily phase is preferably approximately 0.1 to approximately 500 ml/min, more preferably approximately 1 to approximately 400 ml/min and still more preferably approximately 2 to approximately 300 ml/min.

From the viewpoints of minimization of the dispersed particle diameter and the operation costs of the pump, the value calculated by dividing the addition flow rate by the cross-sectional area of the addition port is preferable, that is, the addition linear velocity preferably ranges from approximately 200 to approximately 2,000 m/min, and more preferably from approximately 300 to approximately 1,500 m/min.

It is preferable to perform the jet injection while stirring the water-soluble solvent. In this step, the temperature of the aqueous solvent stirring tank is preferably from 0° C. to 100° C., and more preferably 5° C. to 60° C. Although there may be either one or multiple liquid-feeding ports for the hydrophobic functional ingredient solution, it is preferable to provide from one to five ports. By providing two or more feeding ports, it becomes possible to prepare a microemulsion or a microdispersion comprising multiple kinds of hydrophobic functional ingredients.

When the hydrophobic functional ingredient solution is fed into the stirring tank according to the jet injection method of the invention, it is possible to employ a pump, though it is not always required. In the case of not using a pump, the addition may be conducted by, for example, gravitational dropping.

In the case of using a pump, use can be made of a commercially available feeding pump of the plunger, diaphragm, gear, peristaric or mohno type. From the viewpoints of small pulsation and pressure tolerance, a plunger pump is preferred.

In the jet injection method according to the invention, the hydrophobic functional ingredient solution-feeding port may be a mere cylindrical tube or have a nozzle- or orifice-shape. Although it is preferable that the feeding port has a circular cross-section shape, it may be oval, rectangular or triangular. The feeding port may be made of any materials selected from among various metals, resins, ceramics and the like, though it may be preferable to select a friction-resistant material such as stainless or ceramic.

The stirring velocity (peripheral speed) of the stirrer in the stirring tank is preferably 10 to 1,500 m/min, more preferably 20 to 1,300 m/min and still more preferably 50 to 1,000 m/min. In the case where the stirring is conducted using a pair of stirring blades that are placed at two points in the stirring tank and facing each other at a distance, the stirring velocities of these blades may be either the same or different.

According to the jet injection method of the invention, a microemulsion or a microdispersion can be prepared either by the batch system or the continuous flow system. The continuous flow system is preferred because of being advantageous in mass production.

It is preferable that the jet injection method according to the invention is performed in the submerged jet state wherein the injection is carried out while immersing the hydrophobic functional ingredient solution-feeding port in the aqueous solution. Alternatively, it is also possible to jet the hydrophobic functional ingredient solution in the air close to the aqueous solution surface.

It is preferable in the invention to remove the water-soluble organic solvent having been used after emulsifying or dispersing by the jet injection method. Known examples of the method of removing the solvent include the evaporation method using a rotary evaporator, a flash evaporator or an ultrasonic atomizer; and the membrane separation method using an ultrafiltration membrane or a reverse osmotic membrane. From the viewpoints of the separation efficiency and prevention of the deterioration of the dispersion, the ultrafiltration membrane method and the flash evaporator method may be particularly preferred.

An ultra filter (UF) is a device in which a stock solution (an aqueous solution that is a mixture of water, a high-molecule substance, a low-molecule substance, a colloidal substance, etc.) having been pressurized is injected, separated into two types of solutions, namely, a filtrate (the low-molecule substance) and a concentrate (the high-molecule substance or the colloidal substance) and then taken out.

An ultrafiltration membrane is a typical asymmetrical membrane produced by the Loeb-Sourirajan method. Examples of polymer materials which can be used for ultra-filtration membrane include polyacrylonitrile, polyvinyl chloride-polyacrylonitrile copolymer, polysulfone, polyether sulfone, vinylidene fluoride, aromatic polyamides, cellulose acetate and the like. In recent years, ceramic membranes have been also employed too. Different from the reverse osmosis method and the like, no pretreatment is performed in the ultrafiltration method, which causes fouling, that is, the deposition of the polymers or the like on the membrane surface. Therefore, it is a common practice to wash the membrane with a chemical or hot water at regular intervals. Thus, the membrane material should be resistant to chemicals and heat. There are various membrane module types of ultrafiltration membranes such as the flat membrane type, the tubular type, the hollow fiber type and the spiral type. The performance of ultrafiltration membranes is indicated in fractionation molecular weight and various membranes of 1,000 to 300,000 in fractionation molecular weight are commercially available. Examples of the commercially available membrane modules include, but are not limited to, MICROSER UF (ASAHI KASEI CHEMICALS), capillary type element NTU-3306 (NITTO DENKO, Co.) and the like.

To remove the solvent from the emulsion of the hydrophobic functional component according to the invention, it is particularly preferable from the viewpoint of the solvent-resistance to use a membrane made of polysulfone, polyether sulfone or an aromatic polyamide. With respect to the membrane module form, flat membranes are mainly employed on the laboratory scale, while membranes of the hollow fiber and spiral types are employed industrially. In particular, hollow fiber type membranes are preferred. Although the fractionation molecular weight varies depending on the kind of the active ingredient, membranes having fractionation molecular weight of from 5,000 to 100,000 are commonly used.

Although the available operation temperature range is from 0° C. to 80° C., a temperature range of 10 to 40° C. is particularly preferred by taking the deterioration of the active ingredient into consideration.

Examples of the laboratory-scale ultrafiltration device include ADVANTEC-UHP of the flat membrane module type (ADVANTEC Co., Ltd.), flow-type labo-test unit RUM-2 (NITTO DENKO, Co.) and the like. An industrial plant can be constructed by combining the individual membrane modules in any number and size to satisfy the required capacity. As bench-scale units, RUW-SA (NITTO DENKO, Co.) and the like are marketed.

It is preferable to concentrate the dispersion after removing the solvent. For the concentration, the same method and device as those described with respect to removing the solvent, that is, the evaporation method, the filtration method or the like, and the devices for these methods, can be used. In the concentration, it is also preferable to employ the ultra-filtration method. Although it is preferable to use, if possible, the same membrane as in the solvent removal, ultrafiltration membranes having different fractionation molecular weights may be used if required. It is also possible to conduct the concentration at a different temperature from the solvent removal so as to elevate the concentration efficiency.

The flash evaporator to be used in the invention is a thin film vacuum evaporators, and is comprised, for example, of a liquid reservoir for pooling the dispersion to be concentrated, a spinning column provided with a stirring blade and a heater for evaporation, a condenser for liquefying the evaporated solvent by cooling, a solvent reservoir for pooling the thus liquefied solvent, and a residue reservoir for receiving the solution from which the solvent has been removed. The dispersion to be condensed is fed in portions from the liquid reservoir to the spinning column. The liquid is pressed against the wall to form a thin liquid film by rotating the stirring blade in the spinning column. Then, the wall is heated by the heater so that the solvent is evaporated. Owing to the formation of the thin liquid film, the evaporation efficiency is elevated. Examples of such a flash evaporator include a thin film type evaporator F-70, a thin film type evaporator F-200 and a thin film type evaporator MF-10A (TOKYO RIKA KIKAI), FLASH EVAPO (OKAWARA, Co.) and the like, though the invention is not restricted thereto.

A centrifugal thin-film vacuum evaporator using centrifugal force as a substitute for stirring blades is particularly excellent in concentration efficiency. Examples thereof include EVAPOR (OKAWARA, Co.) and the like.

The emulsion obtained by the preparation method according to the present invention is an oil-in-water type emulsion. From the viewpoint of the transparency of the obtained emulsion, it is preferable that the volume-average particle diameter (median diameter) of the droplets (emulsified particles) is approximately 200 nm or less, more preferably approximately 1 nm to approximately 100 nm and still more preferably approximately 1 nm to approximately 50 nm.

The dispersion obtained by the preparation method according to the invention is an aqueous solid dispersion. From the viewpoint of the transparency of the obtained dispersion, it is preferable that the volume-average particle diameter (median diameter) of the hydrophobic solid microparticles (dispersed particles) is approximately 200 nm or less, more preferably approximately 1 nm to approximately 100 nm and still more preferably approximately 1 nm to approximately 50 nm.

The particle diameter of the emulsified or dispersed particles of the invention can be measured with a commercially available particle diameter distribution measuring device. Known examples of the method of measuring the particle diameter distribution of the emulsified particles in the emulsion or the dispersed particles in the dispersion include the optical microscopy method, the confocal laser microscopy method, the electron microscopy method, the atomic force microscopy method, the static light scattering method, the laser diffraction method, the dynamic light scattering method, the centrifugal precipitation method, the electric pulse measurement method, the chromatography method, the ultrasonic damping method and the like and devices corresponding to the respective principle are commercially available.

From the standpoints of the volume-average particle diameter range in the invention and ease of measurement, the dynamic light scattering method is preferred for measuring the volume-average particle diameter of the invention. Examples of the commercially available measurement devices using dynamic light scattering include Nanotrac UPA (Nikkiso Co., Ltd.), a dynamic light scattering particle diameter distribution measuring device LB-550 (Horiba, Ltd.), a thick type particle diameter analyzer FPAR-1000 (Otsuka Electronics Co., Ltd.) and the like. In the invention, the volume-average particle diameter is measured by using a nano track UPA-EX150 (NIKKISO Co., Ltd.) at 25° C. and the particle diameter and monodispersibility are evaluated. The particle diameter is evaluated in the volume-average particle diameter Mv. The monodispersibility is evaluated based on the value calculated by dividing the volume-average particle diameter Mv by the number-average particle diameter Mn, i.e., Mv/Mn.

The emulsion or dispersion obtained by the invention is a microemulsion or a microdispersion showing extremely little degeneration of the hydrophobic functional ingredient. Therefore, the microemulsion and the microdispersion can be preferably usable for various purposes depending on the kinds of the hydrophobic functional ingredients.

For example, the microemulsion and the microdispersion are usable in foodstuffs, skin externals and medicaments.

That is to say, the foodstuff according to the invention is one wherein the hydrophobic functional ingredient as described above is a functional foodstuff ingredient. The functional foodstuff ingredient may be any of the natural ingredients as cited above that is usable in foods. Examples thereof include, but are not limited to, terpenoids, polyphenols and the like.

The skin external according to the invention is one wherein the hydrophobic functional ingredient as described above is a functional skin external ingredient. The functional skin external may be any of the natural ingredients as cited above that is usable in skin externals. Examples thereof include, but are not limited to, fatty acids, complex lipids, terpenoids and the like.

The medicament according to the invention is one wherein the hydrophobic functional ingredient as described above is a functional medicament ingredient. The functional medicament ingredient may be any of the natural ingredients as cited above that is usable in medicaments. Examples thereof include, but are not limited to, alkaloids, steroids and the like.

### EXAMPLES

Next, the invention will be described in greater detail by referring to the following examples. Unless otherwise noted, all “parts” and “percentages” are by mass.

### Example 1

### Preparation of Emulsion A

The following components were dissolved at 60° C. for 1 hour and then cooled to 25° C. to give aqueous phase A.

| | |
|---|---|
| Sucrose stearate | 0.2 g |
| Decaglyceryl monooleate | 0.2 g |
| Purified water | 300 g |

The following components were dissolved at 40° C. for 1 hour and then cooled to 25° C. to give oily phase A.

| | |
|---|---|
| Haematococcus alga extract (astaxanthin content: 20% by mass) | 0.5 g |
| Tocopherol mixture | 0.1 g |
| Ethanol | 68 g |

The sucrose stearate used above was RYOTO sugar ester S-1670 (HLB=15) manufactured by Mitsubishi-Kagaku Foods Corporation, and the decaglyceryl monooleate used was NIKKOL Decaglyn 1-O (HLB=12) manufactured by Nikko Chemicals Co., Ltd. The Haematococcus alga extract used was ASTOTS-S manufactured by Takeda Shiki Co., Ltd, and the tocopherol mixture was RIKEN E OIL 800 manufactured by Riken Vitamin Co., Ltd. As the ethanol, a reagent grade ethanol manufactured by Wako Pure Chemicals, Inc. was used. As the purified water, an ultrapure water Direct-Q (Millipore Japan) was used.

While stirring the aqueous phase A with a magnetic stirrer at 500 rpm, the oily phase A was submerged and injected into the aqueous phase A by using a plunger pump through a stainless tube of 0.5 mm in diameter at a speed of 80 ml/min. The density and dynamic viscosity of the oily phase were measured at 25° C. As a result, the density of the oily phase measured with a pycnometer was 785 (kg/m³) and the dynamic viscosity of the oily phase measured with a Brookfield E-type viscometer was 1.183 g/m sec. When the Reynolds number was determined, the Reynolds number of the oily phase (oily phase Re) was 2254 under these jet injection conditions. The injection port was positioned on 2 cm below the liquid surface of the aqueous phase A. After continuously adding for 36 seconds, the pump was switched off and simultaneously the injection port was removed from the liquid surface.

Next, the obtained emulsion was collected and named as astaxanthin-containing emulsion A. The oil droplet diameter of the emulsion A measured with Nanotrac UPA (Nikkiso Co., Ltd.) was 120 nm (median diameter).

### Preparation of Emulsion B

Emulsion B was prepared in the same manner as emulsion A except that the following aqueous phase B was used as a substitute for the aqueous phase in emulsion A.

Aqueous phase B

| | |
|---|---|
| Purified water | 300 g |

The oil droplet diameter of the emulsion B determined in the same manner was 135 nm.

### Preparation of Emulsion C

Emulsion C was prepared in the same manner as emulsion A except that the following aqueous phase and oily phase were used as substitutes for the aqueous phase and oily phase in emulsion A.

Aqueous phase C

| | |
|---|---|
| Purified water | 300 g |

Oily phase C

| | |
|---|---|
| Haematococcus alga extract (astaxanthin content: 20% by mass) | 0.5 g |
| Tocopherol mixture | 0.1 g |
| Sucrose stearic acid ester | 0.3 g |
| Decaglyceryl monooleate | 0.3 g |
| Ethanol | 68 g |

The oil droplet diameter of the emulsion C determined in the same manner was 155 nm.

The Reynolds number under the above injection conditions that was determined in the same manner was 2078.

### Preparation of Emulsion D

Emulsion D was prepared using the same compositions for the aqueous phase A and the oily phase A as in emulsion A and in the same manner as in the preparation of emulsion A, except that the oily phase was added at 160 ml/min for an addition time of 18 seconds.

The oil droplet diameter of the emulsion D determined in the same manner was 57 nm.

The Reynolds number under the above injection conditions that was determined in the same manner was 4507.

### Preparation of Emulsion E

Emulsion E was prepared using the same compositions for the aqueous phase A and the oily phase A as in emulsion A and in the same manner as in the preparation of emulsion A, except that the oily phase was added at 240 ml/min for an addition time of 12 seconds.

The oil droplet diameter of the emulsion E determined in the same manner was 25 nm.

The Reynolds number under the above injection conditions that was determined in the same manner was 6761.

### Preparation of Emulsion F

Emulsion F was prepared using the same compositions for the aqueous phase A and the oily phase A as in emulsion A and in the same manner as in the preparation of emulsion A, except that the oily phase was added at 40 ml/min for an addition time of 72 seconds.

The oil droplet diameter of the emulsion F determined in the same manner was 187 nm.

The Reynolds number under the above injection conditions that was determined in the same manner was 1126.

### Preparation of Emulsion G

Emulsion G was prepared using t the same compositions for the aqueous phase A and the oily phase A as in emulsion A and in the same manner as in the preparation of emulsion A, except that the oily phase was added at 30 ml/min for an addition time of 96 seconds.

The oil droplet diameter of the emulsion G determined in the same manner was 356 nm.

The Reynolds number under the above injection conditions that was determined in the same manner was 4507.

### Preparation of Emulsion H

An oily phase having the same composition as the oily phase A was added into an aqueous phase having the same composition as the aqueous phase A without using a pump or an addition tube, and the resulting mixture was treated under a pressure of 200 MPa using a STARBURST MINI (manufactured by Sugino Machine Limited) in three passes to obtain Emulsion H.

The oil droplet diameter of the emulsion H determined in the same manner was 396 nm.

From the above emulsions A to H, ethanol was removed until the ethanol content attained 0.1% by using a laboratory scale ultrafiltration device ADVANTEC-UHP-43K and a polysulfone ultrafiltration membrane Q0500043E (50000 Da). Further, concentration was conducted by using the same device until the astaxanthin content attained 1.0%. Thus, concentrated emulsions A to H were prepared.

The concentrated emulsions A to H were preserved at 50° C. for 28 days and change in the oil droplet diameter and the content of the remaining colorant were measured. The change in the oil droplet diameter was measured as in the measurement of the particle diameter before the preservation, that is, using nano track UPA (NIKKISO Co., Ltd.) at 25° C. The content of the remaining colorant was determined by diluting the emulsions 3000-fold with purified water before and after the preservation, measuring the absorbances thereof and calculating the ratio of the absorbance at 478 nm of the emulsion after the preservation to the absorbance at 478 nm of the emulsion before the preservation.

Table 1 summarizes the data of the oil droplet diameters and the content of the remaining colorant before and after the preservation at 50° C. for 28 days.

Thus, it has been clarified that emulsion according to the invention containing no surfactant in the oily phase and having a higher Reynolds number at injection (1000 or more) exhibited a smaller particle diameter and higher transparency. The emulsions according to the invention also showed favorable results in the change in the oil droplet diameter and the content of the remaining colorant after preserving at 50° C. In contrast thereto, the emulsion C containing the surfactant in the oily phase and the emulsion H prepared by the high-pressure emulsification showed large changes in the oil droplet diameter and undesirable contents of the remaining colorant after the preservation.

### Example 2

### Preparation of Dispersion 2A

As aqueous phase 2A, 300 g of purified water was used.

Oily phase 2A was prepared by dissolving a ceramide solution of the following composition at 60° C. for 1 hour and then cooling to 25° C.

| | |
|---|---|
| Ceramide II | 0.3 g |
| Ceramide IIIB | 0.7 g |
| Ceramide VI | 0.4 g |
| Phytosphingosine hydrochloride | 0.7 g |
| Ethanol | 68 g |

As the above ceramide II, a product of TAKASAGO INTERNATIONAL Corp. was used. As the Ceramide IIIB, Ceramide VI and phytosphingosine hydrochloride, products of Degussa were used. As the ethanol, a reagent grade ethanol manufactured by Wako Pure Chemicals, Inc. was used.

Ceramide dispersion 2A was prepared in the same manner as in the preparation of the emulsion A in Example 1 except that the diameter of the addition tube was changed from 0.5 mm to 1.0 mm and the addition rate of the oily phase was 160 ml/min. The obtained dispersion was collected and the particle diameter of the dispersed particles was measured by using a nano track UPA-EX150 (NIKKISO Co., Ltd.). The volume-average diameter was 28 nm.

The Reynolds number in this jet injection method determined in the same manner as in Example 1 was 1928.

### Preparation of Dispersion 2B

Dispersion 2B was prepared in the same manner as in the dispersion 2A, except that the composition of the oily phase was changed as follows:

| | |
|---|---|
| Ceramide II | 0.3 g |
| Ceramide IIIB | 0.7 g |
| Ceramide VI | 0.4 g |
| Phytosphingosine hydrochloride | 0.7 g |
| Sucrose stearate | 0.3 g |
| Decaglyceryl monooleate | 0.3 g |
| Ethanol | 68 g |

The average particle diameter of the dispersion 2B was 36 nm.

The Reynolds number in this jet injection method determined in the same manner was 1684.

### Preparation of Dispersion 2C

An oily phase having the same composition as that in Dispersion 2A was added into an aqueous phase having the same composition as that in Dispersion 2A without using a pump or an addition tube and then the resulting mixture was treated under a pressure of 200 MPa using a STARBURST MINI (manufactured by Sugino Machine Limited) in five passes to obtain Dispersion 2C.

The oil droplet diameter could not be measured because solid matters separated out immediately after the dispersion.

### Preparation of Dispersion 2D

Dispersion 2D was prepared using the same compositions for the aqueous phase and the oily phase in the dispersion 2A and in the same manner as in the preparation of dispersion 2A, except that the oily phase was jet injected at 320 mL/min into the aqueous phase.

The average particle diameter of the dispersion 2D was 24 nm.

The Reynolds number under the above jet injection that was determined in the same manner was 3856.

### Preparation of Dispersion 2E

Dispersion 2E was prepared using the same compositions for the aqueous phase and the oily phase as in the dispersion 2A and in the same manner as in the preparation of dispersion 2A, except that the oily phase was jet injected at 120 ml/min into the aqueous phase.

The average particle diameter of the dispersion 2E was 44 nm.

The Reynolds number under the above jet injection that was determined in the same manner was 1446.

### Preparation of Dispersion 2F

Dispersion 2F was prepared using the same compositions for the aqueous phase and the oily phase as in the dispersion 2A and in the same manner as in the preparation of dispersion 2A, except that the oily phase was jet injected at 80 ml/min into the aqueous phase.

The average particle diameter of the dispersion 2F was 265 nm.

The Reynolds number under the above jet injection that was determined in the same manner was 962.

### Preparation of Dispersion 2G

Dispersion 2G was prepared using the same compositions for the aqueous phase and the oily phase as in the dispersion 2A and in the same manner as in the preparation of dispersion 2A, except that the diameter of the stainless tube for injecting the oily phase was changed to 0.5 mm and the oily phase was jet injected at 320 ml/min into the aqueous phase.

The average particle diameter of the dispersion 2G was 15 nm.

The Reynolds number under the above jet injection that was determined in the same manner was 7712.

### Preparation of Dispersion 2H

Dispersion 2H was prepared in the same manner as in dispersion 2A except that 0.2 g of phytostenone (UNIFETH manufactured by TOYO HAKKO Co., Ltd.) was added to the oily phase.

The Reynolds number under the above jet injection that was determined in the same manner was 1928.

The ceramide dispersions 2A to 2H as described above were subjected to the ethanol removal and concentrated to 1% by conducting ultrafiltration with the use of the same devices under the same conditions as in Example 1.

The thickened ceramide dispersions 2A to 2H thus obtained were preserved at 50° C. for 28 days, and the conditions of the dispersions were observed and the particle diameters measured. Table 2 shows the results.

The dispersion 2C prepared by the high-pressure dispersion was not well dispersed. Although the dispersion 2B containing the surfactant in the oily phase could be dispersed, it was unstable and showed sedimentation with the passage of time. In contrast thereto, the samples according to the invention sustained the transparency of the liquids and remained stable. Thus, it has been shown that a sample having a larger Reynolds number exhibited smaller particle diameter and higher stability.

All publications, patent applications, and technical standards mentioned in this specification are herein incorporated by reference to the same extent as if such individual publication, patent application, or technical standard was specifically and individually indicated to be incorporated by reference. It will be obvious to those having skill in the art that many changes may be made in the above-described details of the preferred embodiments of the present invention. The scope of the invention, therefore, should be determined by the following claims.

TABLE 1
Emulsification of astaxanthin

| | | | | | Evaluation data | | |
|---|---|---|---|---|---|---|---|
| | | | | | | Particle diameter | Content of residual |
| | | Preparation conditions | | | Particle diameter | after preserving | pigment after |
| | | | | Oily phase | immediately after | for 28 days | preserving for 28 |
| Sample | | Surfactant | Emulsifier device | Re | preparation (nm) | at 50° C. (nm) | days at 50° C. (%) |
| A | Invention | Aqueous phase | Jet-injection | 2254 | 120 | 125 | 91 |
| B | Invention | — | Jet-injection | 2254 | 135 | 134 | 93 |
| C | Comparative | Oily phase | Jet-injection | 2078 | 155 | 269 | 75 |
| D | Invention | Aqueous phase | Jet-injection | 4507 | 57 | 62 | 93 |
| E | Invention | Aqueous phase | Jet-injection | 6761 | 25 | 29 | 90 |
| F | Invention | Aqueous phase | Jet-injection | 1126 | 187 | 195 | 88 |
| G | Comparative | Aqueous phase | Jet-injection | 845 | 356 | 589 | 85 |
| H | Comparative | Aqueous phase | High-pressure injection | — | 396 | 602 | 77 |

TABLE 2
Dispersion of ceramide

| | | Preparation conditions | | | | Evaluation data | |
|---|---|---|---|---|---|---|---|
| | | | | | Oily | Particle diameter | Particle diameter after |
| | | | | Emulsifier | phase | immediately after | preserving for 28 |
| Sample | | Surfactant | Lipid added | device | Re | preparation (nm) | days at 50° C. (nm) |
| 2A | Invention | — | Ceramide | Jet-injection | 1928 | 28 | 30 |
| | | | Phytosphigosine hydrochloride | | | | |
| 2B | Comparative | Oily phase | Ceramide | Jet-injection | 1684 | 36 | Separated |
| | | | Phytosphigosine hydrochloride | | | | |
| 2C | Comparative | — | Ceramide | High-pressure | — | Separated | Separated |
| | | | Phytosphigosine hydrochloride | injection | | | |
| 2D | Invention | — | Ceramide | Jet-injection | 3856 | 24 | 23 |
| | | | Phytosphigosine hydrochloride | | | | |
| 2E | Invention | — | Ceramide | Jet-injection | 1446 | 44 | 47 |
| | | | Phytosphigosine hydrochloride | | | | |
| 2F | Comparative | — | Ceramide | Jet-injection | 962 | 265 | 339 |
| | | | Phytosphigosine hydrochloride | | | | |
| 2G | Invention | — | Ceramide | Jet-injection | 7712 | 15 | 16 |
| | | | Phytosphigosine hydrochloride | | | | |
| 2H | Invention | — | Ceramide | Jet-injection | 1928 | 24 | 25 |
| | | | Phytosphigosine hydrochloride | | | | |
| | | | Phytostenone | | | | |

What is claimed is:

1. A method for preparing an emulsion or a dispersion composed of an oily phase and an aqueous phase by contacting the oily phase with the aqueous phase, the method comprising:
injecting the oily phase, which contains a water-soluble organic solvent and at least one hydrophobic functional ingredient and in which the content of a surfactant is approximately 0.1% by mass or less based on the total mass of the oily phase, into the aqueous phase such that the Reynolds number of the oily phase immediately before contact with the aqueous phase is approximately 1,000 or more.

2. The method for preparing an emulsion or a dispersion according to claim 1, wherein the Reynolds number immediately before contact with the aqueous phase is from approximately 2,000 to approximately 10,000.

3. The method for preparing an emulsion or a dispersion according to claim 1, wherein the addition flow rate of the oily phase is from approximately 0.1 ml/min to approximately 500 ml/min.

4. The method for preparing an emulsion or a dispersion according to claim 1, wherein the volume average particle diameter of the dispersed particles or the emulsified particles in the emulsion or the dispersion ranges from approximately 1 nm to approximately 200 nm.

5. The method for preparing an emulsion or a dispersion according to claim 1, wherein the oily phase contains at least one lipid selected from the group consisting of sphingolipids, phospholipids and sterols.

6. The method for preparing an emulsion or a dispersion according to claim 1, wherein the aqueous phase contains at least one nonionic surfactant having an HLB of 10 to 16.

7. The method for preparing an emulsion or a dispersion according to claim 1, further comprising removing the water-soluble organic solvent from the emulsion or dispersion obtained after the injection.

8. The method for preparing an emulsion or a dispersion according to claim 1, wherein the water-soluble organic solvent is at least one selected from the group consisting of ethanol, propylene glycol and acetone.

9. The method for preparing an emulsion or a dispersion according to claim 1, wherein the water-soluble organic solvent is at least one selected from the group consisting of ethanol and 2-propanol.

10. The method for preparing an emulsion or a dispersion according to claim 1, wherein the water-soluble organic solvent is ethanol.

11. An emulsion or a dispersion prepared by the preparation method according to claim 1.

12. The method for preparing an emulsion or a dispersion according to claim 1, wherein the hydrophobic functional ingredient is a functional foodstuff ingredient.

13. A foodstuff which contains an emulsion or a dispersion prepared by the preparation method according to claim 12.

14. The method for preparing an emulsion or a dispersion according to claim 1, wherein the hydrophobic functional ingredient is a skin external ingredient.

15. A skin external which contains an emulsion or a dispersion prepared by the preparation method according to claim 14.

16. The method for preparing an emulsion or a dispersion according to claim 1, wherein the hydrophobic functional ingredient is a medicinal ingredient.

17. A medicament which contains an emulsion or a dispersion prepared by the preparation method according to claim 16.
